(19) United States
(12) Patent Application Publication
(10) Pub. No.: US 2015/0150925 A1
(43) Pub. Date: Jun. 4, 2015

(54) WOUND HEALING COMPOSITE WITH A FUNCTION OF PROMOTING THE GROWTH OF GRANULATION TISSUES

(71) Applicant: Li Tzu Pao Mei regiman beauty public ltd. company, Kaohsiung City (TW)

(72) Inventor: Hui-Lien Hsieh, Kaohsiung City (TW)

(73) Assignee: Li Tzu Pao Mei regiman beauty public ltd. company, Kaohsiung City (TW)

(21) Appl. No.: 14/094,795

(22) Filed: Dec. 3, 2013

(51) Int. Cl. A61K 36/355; A61K 31/728; A61K 36/254

(52) U.S. Cl. CPC A61K 36/355; A61K 36/254; A61K 31/728

(57) ABSTRACT

A composite with antibacterial and antipruritic functions includes a first substrate extracted from a plant, a second substrate and a base, and the first substrate includes a loniceraiaponica extract and an acanthopanax extract, and both loniceraiaponica extract and acanthopanax extract have a weight percentage ranging from 0.75 to 1.33, and the composite can effectively restrain the itch caused by skin pustules, folliculitis, and mosquito bites, and achieve the moisturization and antipruritic and anti-dander effects for mitigating the symptoms caused by candida albicans and staphylococcus aureus. In addition, this composite has good effects on general skin diseases such as hand eczema (KTPP), eczema, and lip herpes. More importantly, the composite is composed of plant extracts, and thus has a low drug resistance and does not have the side effect of drug allergy.

### FIELD OF THE INVENTION

The present invention relates to a composite with antibacterial and antipruritic functions, and more particularly to the composite with antibacterial and antipruritic functions and composed of plant extracts.

### BACKGROUND OF THE INVENTION

As the issue of environmental pollutions becomes increasingly more seriously, air pollution, water pollution, and food contamination cause various kinds of allergens in our environment, and skin allergy is a common form of allergy.

Statistics show that approximately one-fifth of the present population has skin allergy to different levels. From the medical perspective, skin allergy mainly refers to stimulation of skin by different pollutants such as poor-quality cosmetics, chemicals, pollens, food, and polluted air, wherein symptoms including swelling, itching, peeling and atopic dermatitis of the skin may occur, and the symptoms and levels of the skin allergy vary. Sometimes, symptoms like local tension, numbness and swelling may occur, and other symptoms of skin allergy also include itching, sneezing, runny nose, tears in eyes, rashes, airway obstruction and urticaria or may even jeopardize the patient's life (such as anaphylactic shock).

Atopic dermatitis is a common skin allergy, and atopic dermatitis refers to an abnormal pathogenic change of skin caused by heredity, and symptoms of atopic dermatitis recur and the itch is difficult to stop, and itching is the main characteristic of the atopic dermatitis. Besides the itch, the patient's skin may also swell and have blisters and scabs, and thus the patient's skin is driver than regular skin. The skin of long-term patients becomes thicker and darker. Liquids may be exuded from the affected area of the patient if the patient scratches the affected itching area constantly. For a more serious situation, the skin is infected by bacteria, and pus may be discharged. In the meantime, other allergies may occur easily, and the pathogenesis is still unknown.

At present, a common method of treating atopic dermatitis is to adopt oral antihistamines such as Cyproheptadine, Cetirizine, Loratadine, and Chlorpheniramie to control the itching condition or apply steroids to the affected area for the treatment. Clinically, there are problems such as patients being allergic to western medicines or having difficult to take the oral medicines. If patients apply steroid creams for a long time, the patients' skin may skin and become thinner, and vasodilation of the patients' blood vessels and drug resistance may occur, or the toxicity of the drug may affect the patients' important organs such as liver and kidney. Therefore, non-steroid creams such as Elidel and Protopic are developed and used.

Although Elidel and Protopic can improve the serious issues caused by the conventional steroids, yet Elidel and Protopic are composed of western medicines such as pimecrolimus and tacrolimus which are strictly prohibited for their use for children below two years old. In addition, biological experiments on Elidel and Protopic still have doubts on causing skin cancer and lymphoma, and thus there are still risks of sequelae even though the problems of conventional steroids can be avoided, and it is a main subject for professionals of the related field to develop a medicine with lower toxicity and less side effects to benefit patients with skin diseases.

### DESCRIPTION OF THE PREFERRED EMBODIMENTS

The technical characteristics and contents of the present invention will become apparent with the detailed description of a preferred embodiment as follows:

A preferred embodiment of the present invention comprises a first substrate, a second substrate and a base, extracted from plants. Wherein, the first substrate includes a radix acarthopanacis extract and a loniceraiaponica extract, and the second substrate is a mixture of hyaluronic acid and lactic acid, and the base is water.

Radix acarthopanacis has the effects of maintaining moisture, firming, anti-aging, and anti-oxidation of mucosa.

Wherein, loniceraiaponica has the effects of resisting and curing viruses, fungi, crab itching, swelling, bacteria, external wound infections, cervicitis and various infections and its composition comprises:

### 1. Volatile Oils;

2. Flavonoids and Chlorogenic Acids including luteolin, inositol, chlorogenic acid and isochlorogenic acid in flower stamens;
3. Trierpenoids such as trierpenoid compounds; and
4. Inorganic Elements including 15 trace elements such as Fe, Mn, Cu, Zn, Ti, Sr, Mo, Ba, Ni, Cr, Pb, V, Co, Li, and Ga.

In general, the loniceraiaponica extract such as loniceraiaponica decoction) used for suppressing pathogenic microorganisms (such as Gram positive negative bacteria) has been proven to have a relatively high suppressing effect on influenza virus, orphan virus and herpes through a tissue cell culture process, particularly on Coxsackie virus or Echo virus. In the meantime, biological experiments show that the loniceraiaponica extract can reduce the rival liver damage in mice.

Hyaluronic acid can achieve the effects of promoting the repair of mucosa and maintaining moisture.

Lactic acid can maintain a pH value from 3.5 to 4.5.

Finally, a small quantity of vitamin E can be added into this composite to achieve the effects of improving the skin protection, reducing the dull skin condition, and promoting skin brightness.

In the method of preparing the acanthopanax extract, radix acarthopanacis powder (5 g) is added into ethanol solution (50 mL) with a concentration of 75%, and then heated for an hour for reflux before the solution is filtered, and the filtered solution is heated and evaporated, and pure water (10 mL) is added to the remaining residue to dissolve the residue, and finally the dissolved residue is finally put into a separatory funnel and extracted twice by chloroform (1 mL for each time), and the chloroform solution is combined, heated, and evaporated, and then methanol (1 mL) is added to the remaining residue to dissolve the residue, and the residue is heated and evaporated to obtain an acanthopanax extract.

Loniceraiaponica has a high content of chlorogenic acids, glycosides, flavonoids, and volatile oils, wherein the composition of glycosides includes saponins and iridoid glycosides such as triterpenoid saponins using ivy aglycone as ligand, triterpenoid saponins using oleanolic acid as aglycone, and the composition of flavonoids includes 5-hydroxy- -3,4 (or 7)-trimethoxy flavone, luteolin-7-O-a-D-glucoside, luteolin-7-O-b-D-galactose, quercetin-3-O-b-D-glucoside, and hyperin. In the preparation method, a loniceraiaponica powder (0.1 g) is put into methanol or ethanol (2 mL) to form a loniceraiaponica extract solution, and then dipped into the extract solution at room temperature for 12 hours before it is filtered, and then a methanol solution with a concentration of 70% is added into the loniceraiaponica extract solution until it is quantified to 30 mL, and then the loniceraiaponica extract solution is shaken for 20 minutes before it is heated and baked dry to form a loniceraiaponica extract.

Preferably, the composition of a first preferred embodiment includes a loniceraiaponica extract (35% by weight), an acanthopanax extract (35%), a mixture of hyaluronic acid and lactic acid (10% by weight), and water (20% by weight).

Preferably, the composition of a second preferred embodiment includes a loniceraiaponica extract (30% by weight), an acanthopanax extract (30% by weight), a mixture of hyaluronic acid and lactic acid (10% by weight), and water (30% by weight).

Preferably, the composition of a third preferred embodiment includes a loniceraiaponica extract (40% by weight), an acanthopanax extract (40% by weight), lactic acid (10% by weight), and water (10% by weight).

Preferably, the composition of a fourth preferred embodiment includes a loniceraiaponica extract (30% by weight), an acanthopanax extract (40% by weight), hyaluronic acid (10% by weight), and water (20% by weight).

Preferably, the composition of a fifth preferred embodiment includes a loniceraiaponica extract (40% by weight), an acanthopanax extract (30% by weight), a mixture of hyaluronic acid and lactic acid (10% by weight), and water (20% by weight).

In this preferred embodiment, candida albicans with a concentration of 1.8×10⁴ (CFU/mL) is used as an experiment group, and distilled water added into the candida albicans with a concentration of 1.8×10⁴ (CFU/mL) is used as a control group, and the solutions of these two groups are sat still at room temperature for a period of time, and then the remaining quantity of candida albicans is observed and measured as listed in the experiment data of Table 1.

TABLE 1

| | Original | Remaining Quantity | Remaining Quantity |
|---|---|---|---|
| | Quantity | after 0.5 hour | after 12 hours |
| | (CFU/mL) | (CFU/mL) | (CFU/mL) |
| Control | 1.8 × 10⁴ | 1.8 × 10⁴ | 1.8 × 10⁴ |
| Group | | | |
| Experiment | 1.8 × 10⁴ | <10 | <10 |
| Group | | | |
| Sterilization | | >99.9% | >99.9% |
| Rate | | | |

Wherein, CFU represents the number of colonies per unit volume.

In this preferred embodiment, staphylococcus aureus with a concentration of 1.8×10⁴ (CFU/mL) is used as an experiment group, and distilled water added to the staphylococcus aureus with a concentration of 1.8×10⁴ (CFU/mL) is used as a control group, and the solutions of these two groups are sat still at room temperature for a period of time, and then the remaining quantity of staphylococcus aureus is observed and measured as listed in the experiment data of Table 2.

TABLE 2

| | Original | Remaining Quantity | Remaining Quantity |
|---|---|---|---|
| | Quantity | after 0.5 hour | after 12 hours |
| | (CFU/mL) | (CFU/mL) | (CFU/mL) |
| Control | 1.8 × 10⁴ | 1.8 × 10⁴ | 1.8 × 10⁴ |
| Group | | | |
| Experiment | 1.8 × 10⁴ | <10 | <10 |
| Group | | | |
| Sterilization | | >99.9% | >99.9% |
| Rate | | | |

From Tables 1 and 2, both candida albicans and staphylococcus aureus of the preferred embodiments achieve the sterilization rate over 99.9% within 0.5 hour. In general, candida albicans and staphylococcus aureus are main pathogens causing skin allergies such as candida erosive disease, candidiasis mill rash, and atopic dermatitis. In other words, these preferred embodiments have good effects on the symptoms caused by candida albicans and staphylococcus aureus.

Therefore, the present invention can effectively suppress itching caused by skin pustules, folliculitis or mosquito bites, and achieve the moisturizing and antipruritic and anti-dander effects for mitigating the symptoms caused by candida albicans and staphylococcus aureus. In addition, this composite has good effects on general skin diseases such as hand eczema (KTPP), eczema, and lip herpes. More importantly, the composite of the present invention is composed of plant extracts. Compared with the prior art, the invention has a low drug resistance and does not have the side effect of drug allergy, and the invention can achieve the aforementioned objectives.

While the invention has been described by means of specific embodiments, numerous modifications and variations could be made thereto by those skilled in the art without departing from the scope and spirit of the invention set forth in the claims.

What is claimed is:

1. A composite with antibacterial and antipruritic functions, comprising: a first substrate extracted from a plant, a second substrate and a base, and the first substrate including a loniceraiaponica extract and an acanthopanax extract, and the loniceraiaponica extract and acanthopanax extract have a weight percentage falling within a range from 0.75 to 1.33.

2. The composite with antibacterial and antipruritic functions according to claim 1, wherein the loniceraiaponica extract and the acanthopanax extract have a percentage by weight equal to 1.

3. The composite with antibacterial and antipruritic functions according to claim 1, wherein the second substrate is one selected from hyaluronic acid, lactic acid and a combination of the above.

4. The composite with antibacterial and antipruritic functions according to claim 3, wherein the acanthopanax extract has a percentage by weight falling within a range from 30% to 40%.

5. The composite with antibacterial and antipruritic functions according to claim 4, wherein the loniceraiaponica extract has a percentage by weight falling within a range from 30% to 40%.

6. The composite with antibacterial and antipruritic functions according to claim 1, wherein the second substrate has a percentage by weight equal to 10%.

7. The composite with antibacterial and antipruritic functions according to claim 1, wherein the base is water.

8. The composite with antibacterial and antipruritic functions according to claim 7, wherein the base has a percentage by weight falling within a range from 10% to 20%.
